(19) United States
(12) Patent Application Publication
(10) Pub. No.: US 2024/0091574 A1
(43) Pub. Date: Mar. 21, 2024

(54) NOSTRIL COVERING DEVICE

(71) Applicant: Charles Holland, Baltimore, MD (US)

(72) Inventor: Charles Holland, Baltimore, MD (US)

(21) Appl. No.: 17/947,380

(22) Filed: Sep. 19, 2022

(51) Int. Cl. A62B 23/06; A61M 15/08

(52) U.S. Cl. CPC A62B 23/06; A61M 15/085

(57) ABSTRACT

A nostril covering device, including a first nostril insert, including a first filter body to filter at least one pathogen from air moving therethrough, and a first flap cover movably disposed on at least a portion of the first filter body to cover the first filter body in response to the air moving from outside a boundary of the first flap cover toward the first filter body, and open at least partially away from the first filter body in response to the air moving from the first filter body toward outside the boundary of the first flap cover, a second nostril insert, including a second filter body to filter the at least one pathogen from the air moving therethrough, and a second flap cover movably disposed on at least a portion of the second filter body to cover the second filter body in response to the air moving from outside a boundary of the second flap cover toward the second filter body, and open at least partially away from the second filter body in response to the air moving from the second filter body toward outside the boundary of the second flap cover, and a nose clip removably connected to the first filter body and the second filter body to form a clamp therebetween.

### BACKGROUND

### 1. Field

The present general inventive concept relates generally to a filter, and particularly, to a nostril covering device.

### 2. Description of the Related Art

In late 2019, an unknown disease was discovered and spread rapidly worldwide. The disease has been identified as coronavirus disease 2019 (COVID-19). COVID-19 has been certified by scientists as a pandemic and is likely to become an endemic disease. In the United States, many states have already eased their stay-at-home orders and people are venturing out of the house.

For some people, staying safe while being out of the house poses a dilemma because of the desire to remain protected using a face mask, while consuming food and/or drink in social settings. Typically, the face mask used by many people covers an entirety of the mouth and/or the nose. Unfortunately, the current design of face masks forces a person to constantly remove and then replace their protective covering every time they eat and/or drink, which exposes the person to a pathogen (e.g., virus, bacteria, parasite, fungus), thereby defeating the purpose of the face mask.

Therefore, there is a need for other a nostril covering device that protects nasal passages during breathing while also facilitating eating and/or drinking.

### SUMMARY

The present general inventive concept provides a nostril covering device.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a nostril covering device, including a first nostril insert, including a first filter body to filter at least one pathogen from air moving therethrough, and a first flap cover movably disposed on at least a portion of the first filter body to cover the first filter body in response to the air moving from outside a boundary of the first flap cover toward the first filter body, and open at least partially away from the first filter body in response to the air moving from the first filter body toward outside the boundary of the first flap cover, a second nostril insert, including a second filter body to filter the at least one pathogen from the air moving therethrough, and a second flap cover movably disposed on at least a portion of the second filter body to cover the second filter body in response to the air moving from outside a boundary of the second flap cover toward the second filter body, and open at least partially away from the second filter body in response to the air moving from the second filter body toward outside the boundary of the second flap cover, and a nose clip removably connected to the first filter body and the second filter body to form a clamp therebetween.

The first filter body and the second filter body may expand to adapt to the size of each nostril of the user.

The first nostril insert and the second nostril insert may each further comprise an adjustment body to at least partially deform to adapt to the size of each nostril of the user.

The adjustment body may include a plurality of breathing apertures to facilitate movement of the air through the adjustment body.

The nose clip may include a clip body, a textured edge disposed on at least a portion of an edge of the clip body to cushion the clip body against the nose, and a plurality of insert connection fasteners disposed at a first end and a second end of the clip body to connect the first filter body to the second filter body.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a side perspective view of a nostril covering device, according to an exemplary embodiment of the present general inventive concept; and

FIG. 2 illustrates a bottom perspective view of the nostril covering device as disposed within a nose of a user, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Nostril Covering Device 100
First Nostril Insert 110
First Filter Body 111
First Flap Cover 112
First Adjustment Body 113
First Breathing Apertures 113a
Second Nostril Insert 120
Second Filter Body 121
Second Flap Cover 122
Second Adjustment Body 123
Second Breathing Apertures 123a
Nose Clip 130
Clip Body 131
Textured Edge 132
Insert Connection Fasteners 133

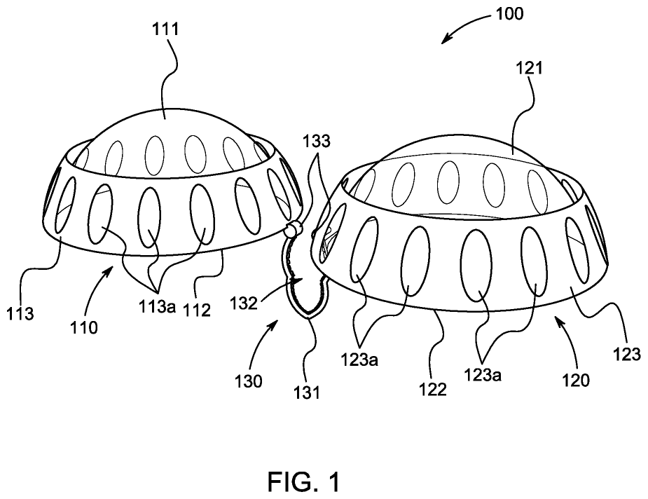

FIG. 1 illustrates a side perspective view of a nostril covering device 100, according to an exemplary embodiment of the present general inventive concept.

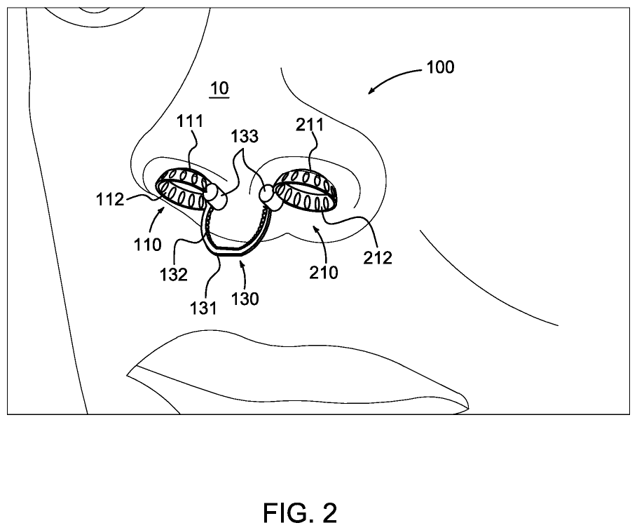

FIG. 2 illustrates a bottom perspective view of the nostril covering device 100 as disposed within a nose 10 of a user, according to an exemplary embodiment of the present general inventive concept.

The nostril covering device 100 may be constructed from at least one of metal, plastic, cloth, and rubber, etc., but is not limited thereto.

The nostril covering device 100 may include a first nostril insert 110, a second nostril insert 120, and a nose clip 130, but is not limited thereto.

The first nostril insert 110 may include a first filter body 111, a first flap cover 112, and a first adjustment body 113, but is not limited thereto.

Referring to FIGS. 1 and 2, the first filter body 111 is illustrated to have a semi-spherical shape. However, the first filter body 111 may be rectangular, circular, conical, triangular, pentagonal, hexagonal, heptagonal, octagonal, or any other shape known to one of ordinary skill in the art, but is not limited thereto. Moreover, the semi-spherical shape may be ideal for each nostril within a nose 10 of a user, such that the first filter body 111 may be rounded and smooth to prevent poking the nose 10 of the user. Also, the first filter body 111 may be sheer and/or a mesh to facilitate movement of air therethrough.

The first filter body 111 and/or the first flap cover 112 may be constructed of a KN95 filter, an N95 filter, an N99 filter, an N100 filter, a P95 filter, a P99 filter, and a P100 filter, but is not limited thereto.

The first filter body 111 may have a predetermined size based on a size of each nostril of the user. For example, the first filter body 111 may have a larger size for an adult compared to a child. As such, the first filter body 111 may be constructed to have a variety of sizes to accommodate nostrils of different users. Alternatively, the first filter body 111 may be elastic and/or at least partially deform (i.e., bend, stretch, expand) to adapt to the size of each nostril of the user. In other words, the first filter body 111 may be a one-size-fits all.

The first flap cover 112 may be movably (i.e., pivotally) disposed on at least a portion of the first filter body 111. The first flap cover 112 may cover at least a portion of a base of the first filter body 111 and/or around an entirety of each edge of the first filter body 111. Moreover, the first flap cover 112 may cover the first filter body 111 in response to the air moving from outside a boundary (e.g., an exterior environment of the nose 10) of the first flap cover 112 toward the first filter body 111. Conversely, the first flap cover 112 may open and/or move at least partially away from the first filter body 111 in response to the air moving from a vertex of the first filter body 111 toward outside the boundary of the first flap cover 112. In other words, the first flap cover 112 opens during exhalation of the air by the user and remains closed to cover the first filter body 111 during inhalation.

Accordingly, the first filter body 111 and/or the first flap cover 112 may filter the air during inhalation to prevent at least one pathogen (e.g., virus, bacteria, parasite, fungus) from entering the nose 10. Conversely, the first flap cover 112 may open during exhalation to facilitate breathing.

Therefore, the first filter body 111 and/or the first flap cover 112 may protect the nose 10 during breathing while allowing the user to eat and/or drink without having to remove a mask and expose the nose 10 to the at least one pathogen.

The first adjustment body 113 may be constructed of plastic, foam, and/or rubber, but is not limited thereto.

The first adjustment body 113 may be circumferentially disposed in a circle around a perimeter of the entirety of the edge of the first filter body 111. The first adjustment body 113 may have a size (e.g., circumference, diameter) greater than the size (e.g., circumference diameter) of the first filter body 111, such that the first adjustment body 113 may surround the first filter body 111. Additionally, the first adjustment body 113 may be elastic and/or be more rigid with respect to the first filter body 111. The first adjustment body 113 may at least partially deform (i.e., bend, stretch, expand) to adapt to the size of each nostril of the user. As such, depending on construction of the first filter body 111 (e.g., predetermined and/or fixed size), the first adjustment body 113 may snugly fit within each nostril of the user. In other words, the first adjustment body 113 may adjust to a size of the nostril to prevent the first filter body 111, the first flap cover 112, and/or the first adjustment body 113 from falling out of the nostril while disposed within the nostril.

The first adjustment body 113 may include a plurality of first breathing apertures 113a, but is not limited thereto.

The plurality of first breathing apertures 113a may be disposed on at least a portion of the first adjustment body 113. Specifically, the plurality of first breathing apertures 113a may be disposed at a distance from each other. Thus, the plurality of first breathing apertures 113a may facilitate movement of the air through the first adjustment body 113.

The second nostril insert 120 may include a second filter body 121, a second flap cover 122, and a second adjustment body 123, but is not limited thereto.

Referring again to FIGS. 1 and 2, the second filter body 121 is illustrated to have a semi-spherical shape. However, the second filter body 121 may be rectangular, circular, conical, triangular, pentagonal, hexagonal, heptagonal, octagonal, or any other shape known to one of ordinary skill in the art, but is not limited thereto. Moreover, the semi-spherical shape may be ideal for each nostril within the nose 10 of the user, such that the second filter body 121 may be rounded and smooth to prevent poking the nose 10 of the user. Also, the second filter body 121 may be sheer and/or a mesh to facilitate movement of the air therethrough.

The second filter body 121 and/or the second flap cover 122 may be constructed of a KN95 filter, an N95 filter, an N99 filter, an N100 filter, a P95 filter, a P99 filter, and a P100 filter, but is not limited thereto.

The second filter body 121 may have a predetermined size based on a size of each nostril of the user. For example, the second filter body 121 may have a larger size for an adult compared to a child. As such, the second filter body 121 may be constructed to have a variety of sizes to accommodate nostrils of different users. Alternatively, the second filter body 121 may be elastic and/or at least partially deform (i.e., bend, stretch, expand) to adapt to the size of each nostril of the user. In other words, the second filter body 121 may be a one-size-fits all.

The second flap cover 122 may be movably (i.e., pivotally) disposed on at least a portion of the second filter body 121. The second flap cover 122 may cover at least a portion of a base of the second filter body 121 and/or around an entirety of each edge of the second filter body 121. Moreover, the second flap cover 122 may cover the second filter body 121 in response to the air moving from outside a boundary (e.g., an exterior environment of the nose 10) of the second flap cover 122 toward the second filter body 121. Conversely, the second flap cover 122 may open and/or move at least partially away from the second filter body 121 in response to the air moving from a vertex of the second filter body 121 toward outside the boundary of the second flap cover 122. In other words, the second flap cover 122 opens during exhalation of the air by the user and remains closed to cover the second filter body 121 during inhalation.

Accordingly, the second filter body 121 and/or the second flap cover 122 may filter the air during inhalation to prevent at least one pathogen (e.g., virus, bacteria, parasite, fungus) from entering the nose 10. Conversely, the second flap cover 122 may open during exhalation to facilitate breathing.

Therefore, the second filter body 121 and/or the second flap cover 122 may protect the nose 10 during breathing while allowing the user to eat and/or drink without having to remove a mask and expose the nose 10 to the at least one pathogen.

The second adjustment body 123 may be constructed of plastic, foam, and/or rubber, but is not limited thereto.

The second adjustment body 123 may be circumferentially disposed in a circle around a perimeter of the entirety of the edge of the second filter body 121. The second adjustment body 123 may have a size (e.g., circumference, diameter) greater than the size (e.g., circumference diameter) of the second filter body 121, such that the second adjustment body 123 may surround the first filter body 121. Additionally, the second adjustment body 123 may be elastic and/or be more rigid with respect to the first filter body 121. The first adjustment body 123 may at least partially deform (i.e., bend, stretch, expand) to adapt to the size of each nostril of the user. As such, depending on construction of the second filter body 121 (e.g., predetermined and/or fixed size), the second adjustment body 123 may snugly fit within each nostril of the user. In other words, the second adjustment body 123 may adjust to a size of the nostril to prevent the second filter body 121, the second flap cover 122, and/or the second adjustment body 123 from falling out of the nostril while disposed within the nostril.

The second adjustment body 123 may include a plurality of second breathing apertures 123a, but is not limited thereto.

The plurality of second breathing apertures 123a may be disposed on at least a portion of the second adjustment body 123. Specifically, the plurality of second breathing apertures 123a may be disposed at a distance from each other. Thus, the plurality of second breathing apertures 123a may facilitate movement of the air through the second adjustment body 123.

The nose clip 130 may include a clip body 131, a textured edge 132, and a plurality of insert connection fasteners 133, but is not limited thereto.

The clip body 131 may have a hinge and/or a spring at a center thereof, such that the clip body 131 may move and/or bend at the center. Moreover, the clip body 131 may be spring biased, such that a first end and a second end of the clip body 131 move toward each other at a point between each end. The clip body 131 may be removably connected to at least a portion of the nose 10 of the user. Additionally, the clip body 131 may clamp around the nose 10 to prevent the first nostril insert 110 and/or the second nostril insert 120 from falling out of the nose 10. In other words, the clip body 131 may form a clamp between the first nostril insert 110 and/or the second nostril insert 120.

The textured edge 132 may include a jagged edge, a plurality of bubbled protrusions extending away from the clip body 131, and a plurality of convex surfaces, but is not limited thereto.

The textured edge 132 may be disposed on at least a portion of an edge of the clip body 131. The textured edge 132 may be soft to at least partially deform (i.e., bend, shrink) in response to an application of force thereto. Thus, the textured edge 132 may provide a cushion (e.g., absorb an impact thereon) within the nose 10 of the user in response to the clip body 131 clamping around a portion of the nose 10, such as a septal nasal cartilage of the nose 10. Also, the textured edge 132 may prevent movement of the first nostril insert 110 and/or the second nostril insert 120 while disposed within the nose 10.

Each of the plurality of insert connection fasteners 133 may include a clamp, a clasp, a clip, a string, a cord, a wire, an adhesive (e.g., tape, glue), a magnet, and/or any combination thereof, but is not limited thereto.

Each of the plurality of insert connection fasteners 133 may be disposed on at least a portion of a first end and/or a second end of the clip body 131 and/or removably connected to at least a portion of the first filter body 111, the first adjustment body 113, the second filter body 121, and/or the second adjustment body 123. The plurality of insert connection fasteners 133 may connect the first nostril insert 110 to the second nostril insert 120.

Therefore, the nostril covering device 100 may protect the nostrils from the at least one pathogen without requiring removal from the nose 10 during eating and/or drinking. Also, the nostril covering device 100 may improve breathability due to the first flap cover 112 and/or the second flap cover 122.

The present general inventive concept may include a nostril covering device 100, including a first nostril insert 110, including a first filter body 111 to filter at least one pathogen from air moving therethrough, and a first flap cover 112 movably disposed on at least a portion of the first filter body 111 to cover the first filter body 111 in response to the air moving from outside a boundary of the first flap cover 112 toward the first filter body 111, and open at least partially away from the first filter body 111 in response to the air moving from the first filter body 111 toward outside the boundary of the first flap cover 112, a second nostril insert 120, including a second filter body 121 to filter the at least one pathogen from the air moving therethrough, and a second flap cover 122 movably disposed on at least a portion of the second filter body 121 to cover the second filter body 121 in response to the air moving from outside a boundary of the second flap cover 122 toward the second filter body 121, and open at least partially away from the second filter body 121 in response to the air moving from the second filter body 121 toward outside the boundary of the second flap cover 122, and a nose clip 130 removably connected to the first filter body 111 and the second filter body 121 to form a clamp therebetween.

The first filter body 111 and the second filter body 121 may expand to adapt to the size of each nostril of the user.

The first nostril insert 110 and the second nostril insert 120 may each further comprise an adjustment body 113/123 to at least partially deform to adapt to the size of each nostril of the user.

The adjustment body 113/123 may include a plurality of breathing apertures 113a/123a to facilitate movement of the air through the adjustment body 113/123.

The nose clip 130 may include a clip body 131, a textured edge 132 disposed on at least a portion of an edge of the clip body 131 to cushion the clip body 131 against the nose, and a plurality of insert connection fasteners 133 disposed at a first end and a second end of the clip body 131 to connect the first filter body 111 to the second filter body 121.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A nostril covering device, comprising:
a first nostril insert, comprising:
a first filter body to filter at least one pathogen from air moving therethrough, and
a first flap cover movably disposed on at least a portion of the first filter body to cover the first filter body in response to the air moving from outside a boundary of the first flap cover toward the first filter body, and open at least partially away from the first filter body in response to the air moving from the first filter body toward outside the boundary of the first flap cover;
a second nostril insert, comprising:
a second filter body to filter the at least one pathogen from the air moving therethrough, and
a second flap cover movably disposed on at least a portion of the second filter body to cover the second filter body in response to the air moving from outside a boundary of the second flap cover toward the second filter body, and open at least partially away from the second filter body in response to the air moving from the second filter body toward outside the boundary of the second flap cover; and
a nose clip removably connected to the first filter body and the second filter body to form a clamp therebetween.

2. The nostril covering device of claim 1, wherein the first filter body and the second filter body expand to adapt to the size of each nostril of the user.

3. The nostril covering device of claim 1, wherein the first nostril insert and the second nostril insert each further comprise:
an adjustment body to at least partially deform to adapt to the size of each nostril of the user.

4. The nostril covering device of claim 3, wherein the adjustment body comprises:
a plurality of breathing apertures to facilitate movement of the air through the adjustment body.

5. The nostril covering device of claim 1, wherein the nose clip comprises:
a clip body;
a textured edge disposed on at least a portion of an edge of the clip body to cushion the clip body against the nose; and
a plurality of insert connection fasteners disposed at a first end and a second end of the clip body to connect the first filter body to the second filter body.
